(19) United States
(12) Patent Application Publication
(10) Pub. No.: US 2023/0248456 A1
(43) Pub. Date: Aug. 10, 2023

(54) SYSTEM AND METHOD FOR DEPTH ESTIMATION IN SURGICAL ROBOTIC SYSTEM

(71) Applicant: Covidien LP, Mansfield, MA (US)

(72) Inventors: Burak Yilmaz, Upton, MA (US); William J. Peine, Ashland, MA (US)

(21) Appl. No.: 18/015,806

(22) PCT Filed: Aug. 5, 2021
(86) PCT No.: PCT/US2021/044593

§ 371 Date: Jan. 12, 2023

Related U.S. Application Data
(60) Provisional application No. 63/067,464, filed on Aug. 19, 2020.

(51) Int. Cl. A61B 34/37; A61B 90/00; A61B 34/00; G06T 7/50; G05D 1/02; G05B 19/4155

(52) U.S. Cl. CPC A61B 34/37; A61B 90/37; A61B 34/77; G06T 7/50; G05D 1/0253; G05B 19/4155; G06T 2207/10028; G05B 2219/50391

(57) ABSTRACT

A surgical robotic system includes a first mobile cart, a control tower coupled to the first mobile cart, and a surgical console coupled to the control tower. The first mobile cart includes a surgical robotic arm and an image capture device actuatable in response to a user input and configured to capture a video of an object in a surgical site. The control tower includes a first controller configured to receive the captured video, determine a speed of the object within the captured video, determine a movement speed of the image capture device, and calculate a distance of the object from the image capture device based on the speed of the object and the movement speed of the image capture device. The surgical console includes a display configured to display the captured video of the surgical site and a user input device configured to generate the user input.

### FIELD

The present disclosure is generally related to a robotic surgical system, in particular, to a system and method for depth estimation of a tissue and/or surgical instrument within the view of a surgical site to control the movement rate of the endoscope camera.

### BACKGROUND

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm.

During procedures with the surgical robotic system, the surgical robotic system lacks the ability to determine distance between the tissue and/or surgical instruments and the endoscope camera. Thus, there is a need for a system to properly determine and estimate such distance information to permit control of the endoscope camera during surgery. Furthermore, there is a need for advanced depth or distance estimation of tissue and instrument to replace or augment 3D views.

### SUMMARY

According to one aspect of the present disclosure, a surgical robotic system includes: a first mobile cart, a control tower coupled to the first mobile cart, and a surgical console coupled to the control tower. The first mobile cart includes a surgical robotic arm and an image capture device actuatable in response to a user input and configured to capture a video of an object in a surgical site. The control tower includes a first controller configured to receive the captured video, determine a speed of the object within the captured video, determine a movement speed of the image capture device, and calculate a depth of the object from the image capture device based on the speed of the object and the movement speed of the image capture device. The surgical console includes a display configured to display the captured video of the surgical site, and a user input device configured to generate the user input.

In aspects, the surgical console may further include a second controller configured to control at least one of directional movement of the image capture device or movement speed of the image capture device.

In aspects, the second controller may further be configured to control the movement speed of the image capture device, and to adjust a scaling factor of the movement speed of the image capture device.

In aspects, the second controller may increase the scaling factor as the distance of the object from the image capture device increases.

In aspects, the second controller may decrease the scaling factor as the distance of the object from the image capture device decreases.

In aspects, the second controller may be further configured to control the movement speed of the image capture device to maintain a ratio between a movement speed of the user input device and the movement speed of the object within the captured video.

In aspects, the surgical robotic system may further include a second mobile cart having a second surgical robotic arm and a surgical instrument actuatable in response to a user input and configured to treat tissue.

In aspects, the surgical console may further include a second controller configured to perform at least one of the following: control the surgical instrument or register the surgical instrument position based on the distance of the surgical instrument from the image capture device.

According to another aspect of the present disclosure, a surgical robotic system includes a first mobile cart, a control tower coupled to the first mobile cart, and a surgical console coupled to the control tower. The first mobile cart includes a surgical robotic arm and an image capture device actuatable in response to a user input and configured to capture a video of a surgical site. The control tower includes a first controller configured to: track movement speed of a distal end portion of the surgical instrument by the image capture device and calculate a distance of the distal end portion of the surgical instrument from the image capture device based on the tracked movement speed and the movement speed. The surgical console includes a display configured to display the captured video of the surgical site, and a user input device configured to generate the user input.

In aspects, the surgical console may further include a second controller configured to register a position of the instrument relative to the captured video based on the distance of the distal end portion of the surgical instrument from the image capture device.

According to another aspect of the present disclosure, a method of determining a distance of an object from an image capture device of a surgical robotic system includes capturing a video of an object in a surgical site; determining speed of the object within the captured video; determining a movement speed of the image capture device; calculating a distance of the object from the image capture device based on the speed of the object and the movement speed of the image capture device; and controlling at least one of directional movement of the image capture device or the movement speed of the image capture device based on the calculated distance of the object from the image capture device.

In aspects, controlling directional movement of the image capture device may include controlling zooming, panning, autofocus, or auto exposure.

In aspects, controlling the movement speed of the image capture device may include adjusting a scaling factor of the actual movement speed of the image capture device.

In aspects, the scaling factor may increase as the distance of the object from the image capture device increases.

In aspects, the scaling factor may decrease as the distance of the object from the image capture device decreases.

In aspects, controlling the movement speed of the image capture device may maintain a ratio between a movement speed of the user input device and the movement speed of the object within the captured video.

In aspects, the method may further include controlling at least one component of the surgical instrument based on the distance of the surgical instrument from the image capture device.

In aspects, the method may further include registering the surgical instrument within the captured video based on the distance of the surgical instrument from the image capture device.

### BRIEF DESCRIPTION OF DRAWINGS

Various embodiments of the present disclosure are described herein with reference to the drawings wherein:

FIG. 1 is a schematic illustration of a surgical robotic system including a control tower, a console, and one or more surgical robotic arms according to the present disclosure;

FIG. 2 is a perspective view of a surgical robotic arm of the surgical robotic system of FIG. 1 according to the present disclosure;

FIG. 3 is a perspective view of a setup arm with the surgical robotic arm of the surgical robotic system of FIG. 1 according to the present disclosure;

FIG. 4 is a schematic diagram of a computer architecture of the surgical robotic system of FIG. 1 according to the present disclosure;

FIG. 5 is an exemplary view of a video feed at a surgical site; and

FIG. 6 is a flow chart illustrating a method according to the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, or a server system.

As will be described in detail below, the present disclosure is directed to a surgical robotic system, which includes a surgical console, a control tower, and one or more movable carts having a surgical robotic arm coupled to a setup arm. The control tower includes a controller, which is configured to determine depth or distance information of a tissue and/or a surgical instrument based on a tracked speed of the tissue and/or surgical instrument and the known movement of the surgical robotic system.

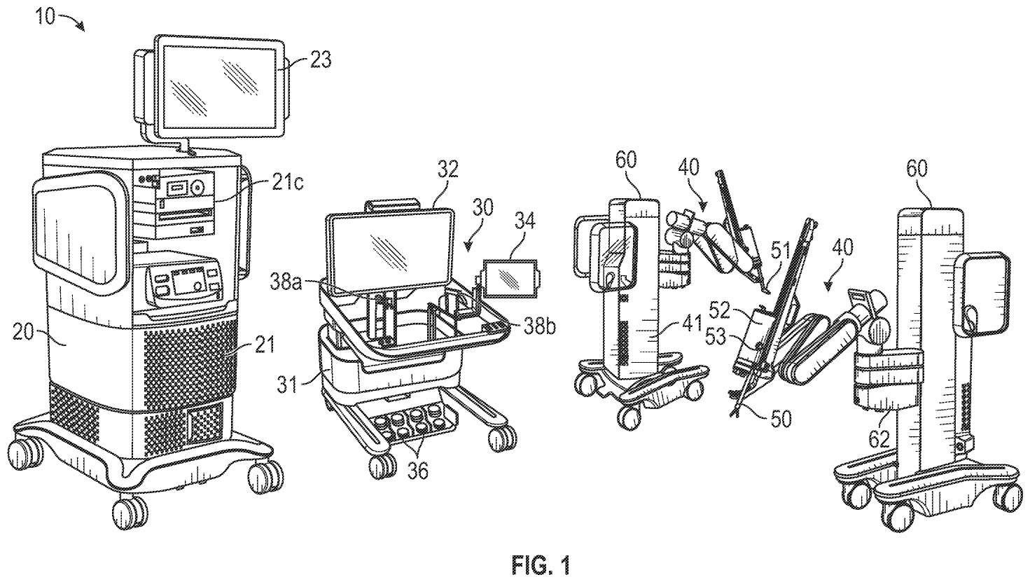

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

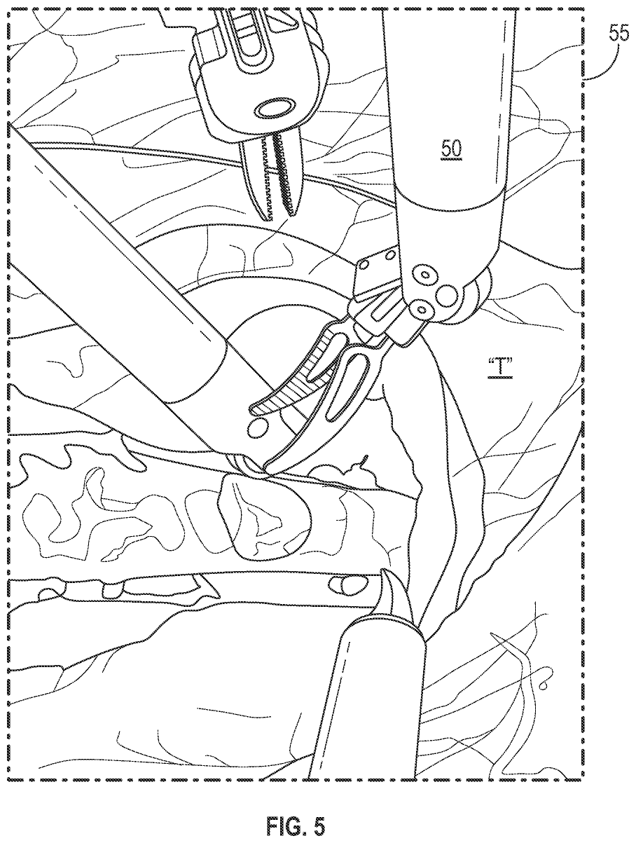

The surgical instrument 50 is configured for use during minimally invasive surgical procedures. In embodiments, the surgical instrument 50 may be configured for open surgical procedures. In embodiments, the surgical instrument 50 may be an endoscope, such as an camera 51, configured to provide a video feed 55 for the user (FIG. 5). In further embodiments, the surgical instrument 50 may be an electrosurgical forceps configured to seal tissue by compression tissue between jaw members and applying electrosurgical current thereto. In yet further embodiments, the surgical instrument 50 may be a surgical stapler including a pair of jaws configured to grasp and clamp tissue whilst deploying a plurality of tissue fasteners, e.g., staples, and cutting stapled tissue.

One of the robotic arms 40 may include a camera 51 configured to capture video of the surgical site. The surgical console 30 includes a first display 32, which displays a video feed 55 of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of hand controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the hand controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

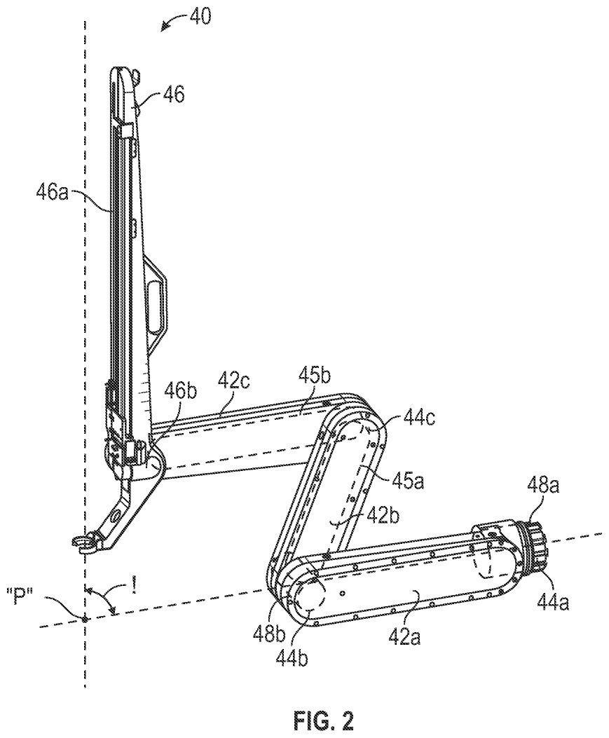

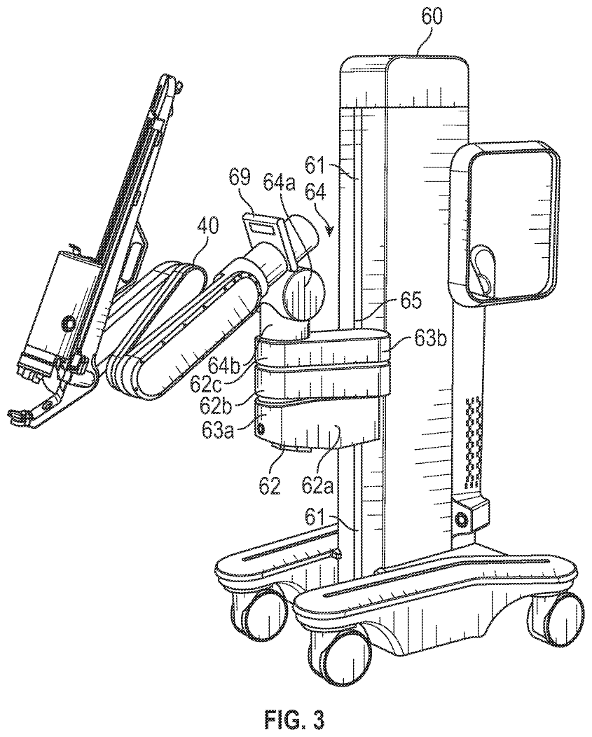

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference to FIG. 2, the robotic arm 40 also includes a holder 46 defining a second longitudinal axis and configured to receive an IDU 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 also includes a plurality of manual override buttons 53 disposed on the IDU 52 and the setup arm 62, which may be used in a manual mode. The user may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

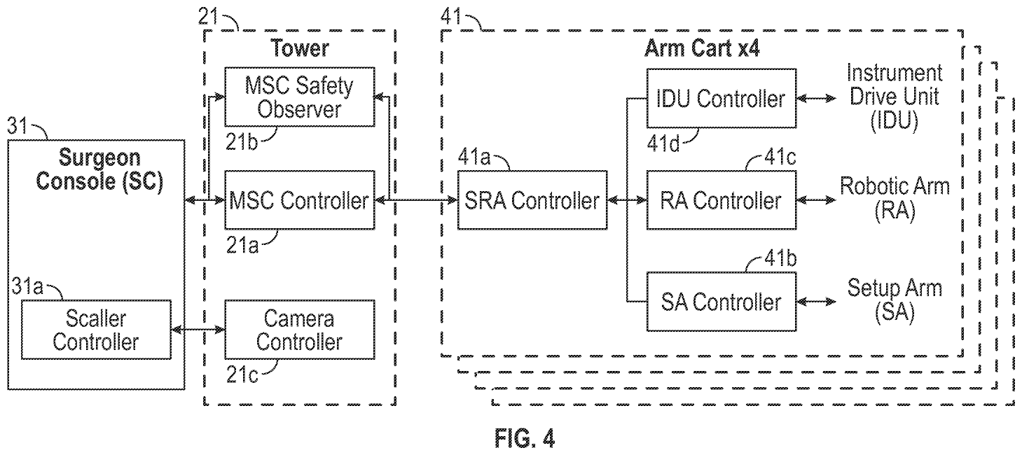

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled as follows. Initially, a pose of the hand controller controlling the robotic arm 40, e.g., the hand controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the hand controller 38a is then scaled by a scaling function executed by the controller 21a. In embodiments, the coordinate position is scaled down and the orientation is scaled up by the scaling function. In addition, the controller 21a also executes a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

With reference to FIGS. 4 and 5, the computer 21 of the control tower 20 further includes a camera controller 21c, which is configured to receive the video feed 55 from the camera 51. The camera controller 21c further analyzes the video feed 55 to determine a speed of the instrument 50 within the video feed 55 as well as the speed of the camera 51. The video feed 55 is configured to be displayed on the first display 32. The speed of the surgical instrument 50 may be determined through optical flow which analyzes the motion of the surgical instrument 50 within the video feed 55 across multiple frames by comparing the motion and/or position of the tissue “T” and/or surgical instrument 50 and other objects in the video feed 55 in between frames. Thus, stationary objects, such as tissue, appear as moving in the video feed 55 due to the movement of the camera 51, thus, the image motion allows the camera controller 21c to determine the speed of movement of the camera 51. In some instances, the camera controller 21c may determine the motion of multiple instrument 50 and any other portion of the surgical robotic system 10 or objects that may be present in the surgical site captured within the video feed 55.

The camera controller 21c further receives actual movement inputs (e.g., position and speed) of the camera 51 attached to the robotic arm 40 based on the calculated movement command communicated to one or more of the actuators 48a and 48b in the robotic arm 40 and the actual joint positions of the actuators 48a and 48b in the robotic arm 40. The camera controller 21c determines a distance or depth of the tissue “T” and/or surgical instrument 50 from the camera 51 based on the speed of the tissue “T” and/or surgical instrument 50 within the video feed 55 and the actual movement of the camera 51.

The camera controller 21c is further configured to track the movement of a distal end portion of the instrument 50 during movement of the instrument 50 and/or movement of the camera 51. The camera controller 21c determines a depth or distance of the distal end portion of the instrument 50 from the camera 51 based on the actual speed of instrument 50 and/or camera 51 and the tracked movement of the distal end portion of the instrument 50. Thus, the camera controller 21c is provided movement speed and/or position of the instrument 50 and the camera 51, and utilizes both to determine the distance of the instrument 50 from the camera 51.

The computer 31 of the surgical console further includes a scaler controller 31a, which is configured to output a scaling factor based on the distance of the tissue “T” and/or surgical instrument 50 from the camera 51. The scaling factor may be used to adjust the image controls of camera 51, movement of the components of the surgical instrument 50, movement of the camera 51.

In adjusting the speed of the actual movement of the camera 51, the scaler controller 31a may increase or decrease the speed of the actual movement of the camera 51. The scaler controller 31a decreases the speed of the actual movement of the camera 51 as the distance between the tissue “T” or surgical instrument 50 and the camera 51 decreases, thereby allowing the camera 51 to move more slowly and precisely when working close to the tissue “T” or surgical instrument 50. The scaler controller 31a increases the speed of the actual movement of the camera 51 as the distance between the tissue “T” or surgical instrument 50 and the camera 51 increases, thereby allowing the camera 51 to move more quickly to cover more distance within the video feed 55. The speed of the actual movement of camera 51 is adjusted by applying a scaling factor to the calculated movement command communicated to the robotic arm 40. As a result of the speed of the actual movement of the camera 51 being adjusted, the surgical robotic system 10 maintains a constant rate of speed for the video feed 55. In particular, the surgical robotic system 10 maintains a ratio between the movement speed of the handle controllers 38a controlling the camera 51 and the speed of the objects in the captured video (for image capture device) and/or to maintain a ratio between the movement speed of the handle controllers 38b controlling the instrument 50 and the speed of the instrument 50 in the captured video). In embodiments, the ratio that is maintained may be constant. In other embodiments, the maintained ratio may be variable. The ratio may be set automatically by the surgical robotic system 10 or the ratio may be user-selectable, i.e., set manually by the user. Thus, the surgical robotic system 10 provides a proportionate relationship between camera 51 attached to the robotic arm 40 and the handle controllers 38a and 38b during operation.

The ratio is perceived by the user looking at the first display 32, which displays a video feed 55 very differently when the camera 51 is close to the object and when it is far away. Thus, the surgical robotic system 10 provides an estimated depth information into the video feed 55 and adjusts the movement speed of the object in space using the scaling factor in order to maintain the movement ratio. The ratio may also be applied to the motion of the camera 51 itself. The image movement of the video feed 55 supplied by the camera 51 in space may be adjusted in order to make the image view move at a constant ratio of the handle controllers 38a in space.

In adjusting the scaling factor (i.e., ratio) of the image controls of camera 51, the scaler controller 31a may adjust by a scaling factor the camera control algorithms based on the determined depth that affect image movement, such as zooming, panning, autofocus, auto exposure, and any other camera control algorithms that are suitable to maintain an optimal visual experience for the clinician.

In adjusting the scaling factor (i.e., ratio) of the components of the instrument 50, the scaler controller 31a is configured to decrease the actuation speed of the components of the instrument 50 as the distance between the tissue “T” or surgical instrument 50 and the camera 51 decreases and increases the actuation speed of the components of the instrument 50 as the distance between the tissue “T” or surgical instrument 50 and the camera 51 increases. The actuation speed of the components is adjusted by applying a scaling factor to the known actuation speed of the component of the instrument 50. The scaler controller 31a may be used to apply a scaling factor to other components and systems of the surgical robotic system 10. Maintaining proportionate relationship between the motion of the instrument 50 in the video feed 55 and motion of the handle controller 38a in the space also improves the visual experience for the clinician.

The scaler controller 31a is further configured to register the position of the instrument 50. The scaler controller 31a receives the distance of the distal end portion of the instrument 50 from the camera 51 and provides a coordinate position of the instrument 50 within the video feed 55, thereby providing the surgical robotic system 10 the ability to prevent collision between multiple instrument(s) 50, camera(s) 51, and tissue “T”.

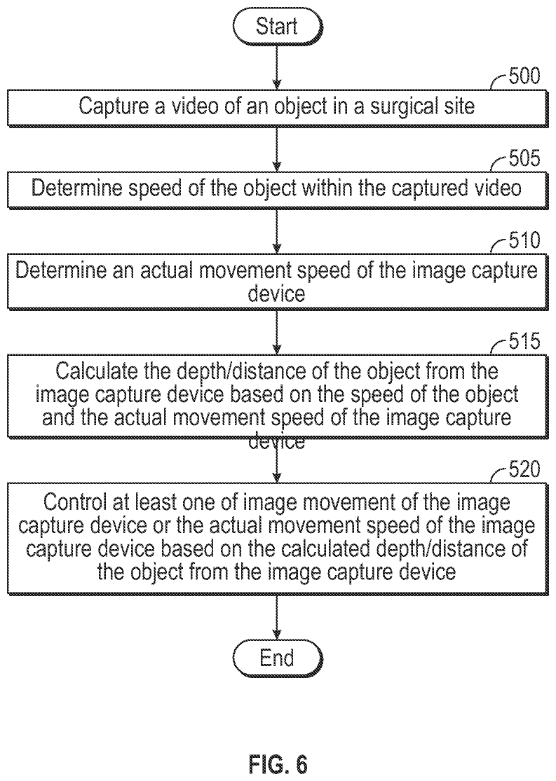

With reference to FIGS. 5 and 6, during operation, the camera 51 coupled to a surgical arm is inserted in the surgical site and, at step 500, captures a video feed 55 of the tissue “T” and/or surgical instrument 50 in the surgical site. Once the video feed 55 of the surgical site is captured, at step 505, the camera controller 21c determines a speed of the tissue “T” and/or surgical instrument 50 in the video feed 55. The camera controller 21c, at step 510, receives the actual movement speed of the camera 51 based on the calculated movement command. The camera controller 21c, at step 515, calculates the distance of the tissue “T” and/or surgical instrument 50 from camera 51 based on the speed of the tissue “T” and/or surgical instrument 50 and the movement speed of the camera 51. Once the distance of the tissue “T” and/or surgical instrument 50 from camera 51, at step 520, the scaler controller 31a controls one of: directional movement of camera 51, speed of the components of the instrument 50, speed of the movement of the camera 51, and/or position of the instrument 50 within the video feed 55.

While the present disclosure provides for depth estimation performed using the actual and camera motions that are observed, and their relation relative to reach other, it is envisioned that depth of the instrument may be estimated using other techniques, such as by calculating the pixel-size of the instrument in the image, and using that data, together with camera focal length and the actual size of the instrument.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical robotic system comprising:
a first mobile cart including a surgical robotic arm and an image capture device actuatable in response to a user input and configured to capture a video of an object in a surgical site;
a control tower coupled to the first mobile cart, the control tower including:
a first controller configured to:
receive the captured video;
determine a movement speed of the object within the captured video;
determine a movement speed of the image capture device; and
calculate a distance of the object from the image capture device based on the movement speed of the object and the movement speed of the image capture device; and
a surgical console coupled to the control tower, the surgical console including:
a display configured to display the captured video of the surgical site; and
a user input device configured to generate the user input.

2. The surgical robotic system according to claim 1, wherein the surgical console further includes a second controller configured to control at least one of image movement of the image capture device or the movement speed of the image capture device.

3. The surgical robotic system according to claim 2, wherein the second controller is further configured to adjust a scaling factor of at least one of image movement of the image capture device or the movement speed of the image capture device.

4. The surgical robotic system according to claim 3, wherein the second controller increases the scaling factor as the distance of the object from the image capture device increases.

5. The surgical robotic system according to claim 3, wherein the second controller decreases the scaling factor as the distance of the object from the image capture device decreases.

6. The surgical robotic system according to claim 2, wherein the second controller is further configured to maintain a ratio between a movement speed of the user input device and the movement speed of the object within the captured video.

7. The surgical robotic system according to claim 1, further comprising:
a second mobile cart including a second surgical robotic arm and a surgical instrument actuatable in response to a user input and configured to treat tissue.

8. The surgical robotic system according to claim 7, wherein the surgical console further includes a third controller configured to perform at least one of the following:
control the surgical instrument; or
register the surgical instrument position based on the distance of the surgical instrument from the image capture device.

9. A surgical robotic system comprising:
a first mobile cart including a surgical robotic arm and an image capture device actuatable in response to a user input and configured to capture a video of a surgical site;
a control tower coupled to the first mobile cart, the control tower including:
a first controller configured to:
track movement speed of a distal end portion of the surgical instrument based on the video captured by the image capture device; and
calculate a distance of the distal end portion of the surgical instrument from the image capture device based on the tracked movement speed and the movement speed; and
a surgical console coupled to the control tower, the surgical console including:
a display configured to display the captured video of the surgical site; and
a user input device configured to generate the user input.

10. The surgical robotic system according to claim 9, wherein the surgical console further includes a second controller configured to register a position of the instrument based on the distance of the distal end portion of the surgical instrument from the image capture device.

11. A method of determining distance of an object from an image capture device of a surgical robotic system, the method comprising:
capturing a video of an object in a surgical site;
determining a speed of the object within the captured video;
determining a movement speed of the image capture device;
calculating a distance of the object from the image capture device based on the speed of the object and the actual movement speed of the image capture device; and
controlling at least one of image movement of the image capture device or the movement speed of the image capture device based on the calculated distance of the object from the image capture device.

12. The method according to claim 11, wherein controlling the image movement of the image capture device includes controlling zoom, panning, autofocus, or auto exposure.

13. The method according to claim 12, wherein controlling the movement speed of the image capture device includes adjusting a scaling factor of the actual movement speed of the image capture device.

14. The method according to claim 13, wherein the scaling factor increases as the distance of the object from the image capture device increases.

15. The method according to claim 14, wherein the scaling factor decreases as the distance of the object from the image capture device decreases.

16. The method according to claim 12, wherein controlling the actual movement speed of the image capture device maintains a ratio between a movement speed of the user input device and the movement speed of the object within the captured video.

17. The method according to claim 11, further comprising controlling at least one component of the surgical instrument based on the distance of the surgical instrument from the image capture device.

18. The method according to claim 11, further comprising registering the surgical instrument within the captured video based on the distance of the surgical instrument from the image capture device.
